(19) United States
(12) Patent Application Publication
(10) Pub. No.: US 2013/0280820 A1
(43) Pub. Date: Oct. 24, 2013

(54) BIOMARKERS FOR PSORIASIS

(71) Applicant: MARIBEL BEAUMONT, Redwood City, CA (US)

(72) Inventor: MARIBEL BEAUMONT, Redwood City, CA (US)

(73) Assignee: SCHERING CORPORATION, KENILWORTH, NJ (US)

(21) Appl. No.: 11/610,939

(22) Filed: Dec. 14, 2006

Related U.S. Application Data
(60) Provisional application No. 60/751,191, filed on Dec. 16, 2005.

(51) Int. Cl. G01N 33/68; H01J 49/00

(52) U.S. Cl. CPC G01N 33/6881; H01J 49/0036
USPC 436/501; 250/282

(57) ABSTRACT

A group of polypeptides that are modulated in a psoriatic sample as compared to a normal sample is provided. These polypeptides can be used as biomarkers for diagnosis and monitoring treatment of psoriasis.

This filing claims benefit of U.S. Provisional Patent Application No. 60/751,191, filed Dec. 16, 2005, which is hereby incorporated by reference in its entirety.

### FIELD OF THE INVENTION

The present invention relates to biological markers for skin inflammation, more particularly, psoriasis. More specifically, the present invention relates to the use of such markers to diagnose and treat psoriasis, monitor progression of the disease, evaluate therapeutic interventions, and screen candidate drugs in a clinical or preclinical setting.

### BACKGROUND OF THE INVENTION

The skin serves as an important boundary between the internal milieu and the environment, preventing contact with potentially harmful antigens. In the case of antigen/pathogen penetration, an inflammatory response is induced to eliminate the antigen. This response leads to a dermal infiltrate that consists predominantly of T cells, polymophonuclear cells, and macrophages (see, e.g., Williams and Kupper (1996) Life Sci., 58:1485-1507.) Normally, this inflammatory response, triggered by the pathogen, is under tight control and will be halted upon elimination of the pathogen.

In certain cases, this inflammatory response occurs without external stimuli and without proper controls, leading to cutaneous inflammation. Cutaneous inflammation, the result of the cellular infiltrate noted above as well as the secreted cytokines from these cells, encompasses several inflammatory disorders such as cicatricial pemphigoid, scleroderma, hidradenitis suppurativa, toxic epidermal necrolysis, acne, osteitis, graft vs. host disease (GvHD), pyroderma gangrenosum, and Behcet's Syndrome (see, e.g., Willams and Griffiths (2002) Clin. Exp. Dermatol., 27:585-590). The most common form of cutaneous inflammation is psoriasis.

Psoriasis is characterized by T cell mediated hyperproliferation of keratinocytes coupled with an inflammatory infiltrate. The disease has certain distinct by overlapping clinical phenotypes including chronic plaque lesions, skin eruptions, and pustular lesions (see, e.g., Gudjonsson, et al. (2004) Clin Exp. Immunol. 135:1-8). Approximately 10% of psoriasis patients develop arthritis. The disease has a strong but complex genetic predisposition, with 60% concordance in monozygotic twins.

The typical psoriatic lesion is a well defined erythematous plaque covered by thick, silvery scales. The inflammation and hyperproliferation of psoriatic tissue is associated with a different histological, antigenic, and cytokine profile than normal skin. Among the cytokines associated with psoriasis are: TNFα, IL-18, IL-15, IL-12, IL-7, IFNγ, IL-17A and IL-23 (see, Gudjonsson, et al., supra).

To date, monitoring and diagnosis of psoriasis has been hampered by lack of knowledge of the molecular changes between normal and psoriatic samples. The present invention fills this unmet need by providing a set of biomarkers that are differentially modulated in normal versus psoriatic samples.

### SUMMARY OF THE INVENTION

The invention is based, in part, upon the discovery that certain polypeptides are differentially modulated when psoriatic tissue is compared to normal tissue. The present invention contemplates a combination comprising a plurality of isolated polypeptides of Table 1, wherein the polypeptides are differentially expressed in a sample from a first subject suffering from psoriasis as compared to a sample from a second subject not suffering from psoriasis. In certain embodiments, the combination comprises 1, 2, 3, 4, 5, 6, 7, 8, 9, 10 or more, or all, of the polypeptides of Table 1; the sample is biological sample, including plasma; the first and second subjects are mammals, including primates and humans. In a further embodiment, the levels of the polypeptides of Table 1 are determined by 2D DIGE/mass spectrometry analysis or by immunoassay, e.g. ELISA.

The present invention provides method of diagnosing psoriasis in a subject, the method comprising: a) obtaining one or more biological samples from the subject; b) determining the level(s) of one or more of the polypeptides of Table 1 in the one or more biological samples; and c) comparing the level(s) of the one or more of polypeptides to a reference value. In some embodiments the reference value is the level of the one or more polypeptides of Table 1 in a biological sample from one or more non-psoriatic subjects. In other embodiments the reference value is the level in a biological sample from one or more psoriatic subjects. In some embodiments the one or more polypeptides of Table 1 comprise 1, 2, 3, 4, 5, 6, 7, 8, 9, 10 or more, or all, of the polypeptides of Table 1. In other embodiments the biological sample is a body fluid; including plasma.

The present invention encompasses a method of monitoring the progression of psoriasis in a subject, the method comprising: a) obtaining a first biological sample from the subject; b) measuring the level of one or more polypeptides of Table 1 in the first sample; c) obtaining a second biological sample from the subject; d) measuring the level of the one or more polypeptides of Table 1 in the second sample; and e) comparing the levels in the first and second samples to each other (i.e. comparing the level in the first sample to the level in the second sample). In some embodiments the one or more polypeptides of Table 1 comprise 1, 2, 3, 4, 5, 6, 7, 8, 9, 10 or more, or all, of the polypeptides of Table 1.

In another embodiment the first biological sample from the subject is obtained at time t₀, and the second biological sample from the subject is obtained at time t₁, and t₀ is before t₁. In yet another embodiment, additional first and second samples are obtained at a series of time points.

In another embodiment the subject is treated with a treatment for psoriasis after t₀ but before t₁ (i.e. between the times when the first and second samples are obtained). In one embodiment the levels of one or more polypeptides of Table 1 before and after treatment are compared to determine whether the treatment (therapeutic intervention) is consistent with an improvement in the subject's psoriasis, e.g. as reflected in a reduction in PASI score (e.g. to PASI<10). In various embodiments the treatment is a treatment of known efficacy, or it may be an experimental treatment. In embodiments involving treatments with established efficacy the method of monitoring the progression of psoriasis of the present invention may be used to guide further decisions in the course of treatment of the subject, i.e. to manage the treatment of the subject. Such management may include decisions to alter dosing, administration scheduling, adding other therapeutic methods, switching to a different therapeutic approach, or discontinuing treatment altogether. In one embodiment, such management of treatment is the selection of one of a plurality of potential therapeutic regimens for the treatment of psoriasis based on the level(s) of the one or more polypeptides of Table 1 in that particular subject. Such selection of subgroups of psoriatic subjects for specific therapeutic regimens may be used to target a therapeutic regimen only to those subjects in which it is likely to be efficacious.

In embodiments in which an experimental treatment is used, the method of monitoring the progression of psoriasis of the present invention may be used to determine whether the experimental treatment (therapeutic intervention) is efficacious. In one embodiment, the method of monitoring is used to determine whether a proposed therapeutic agent (e.g. a compound) is efficacious in the treatment of psoriasis, for example in preclinical studies or in a clinical trial.

In another aspect the present invention relates to kits to enable detection of one or more of the polypeptides of Table 1. In one embodiment the kit comprises a solid support comprising at least two capture reagents, such as antibodies or antigen binding fragments thereof, that each bind to different polypeptides of Table 1, and instructions for use of the solid support to detect the different polypeptides of Table 1.

### DETAILED DESCRIPTION

As used herein, including the appended claims, the singular forms of words such as “a,” “an,” and “the” include their corresponding plural references unless the context clearly dictates otherwise. All references cited herein are incorporated by reference to the same extent as if each individual publication, patent application, or patent, was specifically and individually indicated to be incorporated by reference.

### I. DEFINITIONS

“Activity” of a molecule may describe or refer to binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. Treatment encompasses methods using a purified immune cell, e.g., in a mixed cell reactions or for administration to a research, animal, or human subject. The invention contemplates treatment with a cell, a purified cell, a stimulated cell, a cell population enriched in a particular cell, and a purified cell. Treatment further encompasses situations where an administered reagent or administered cell is modified by metabolism, degradation, or by conditions of storage.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, including selenomethionine, as well as those amino acids that are modified after incorporation into a polypeptide, e.g., hydroxyproline, O-phosphoserine, O-phosphotyrosine, gamma-carboxyglutamate, and cystine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetic refers to a chemical compound that has a structure that is different from the general chemical structure of an amino acid, but that functions in a manner similar to a naturally occurring amino acid. Amino acids may be referred to herein by either their commonly known three letter symbols or by their one-letter symbols.

“Binding composition” refers to a molecule, small molecule, macromolecule, antibody, a fragment or analogue thereof, or soluble receptor, capable of binding to a target. “Binding composition” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, which is capable of binding to a target. “Binding composition” may also refer to a molecule in combination with a stabilizer, excipient, salt, buffer, solvent, or additive, capable of binding to a target. “Binding” may be defined as an association of the binding composition with a target where the association results in reduction in the normal Brownian motion of the binding composition, in cases where the binding composition can be dissolved or suspended in solution.

A “biological marker” or “biomarker” as used herein, is “a characteristic that is objectively measured and evaluated as an indicator of normal biologic processes, pathogenic processes, or pharmacological responses to therapeutic interventions.” (See, e.g., NIH Biomarker Definitions Working Group (1998)). Biological markers can also include patterns or ensembles of characteristics indicative of particular biological processes (“panel of markers”). The marker measurement can be increased or decreased to indicate a particular biological event or process. In addition, if a marker measurement typically changes in the absence of a particular biological process, a constant measurement can indicate occurrence of that process.

As used herein, the term “biological sample” means any biological substance, including but not limited to blood (including whole blood, leukocytes prepared by lysis of red blood cells, peripheral blood mononuclear cells, plasma and serum), sputum, urine, semen, cerebrospinal fluid, bronchial aspirate, sweat, feces, synovial fluid, cells, and whole or manipulated tissue.

“Bispecific antibody” generally refers to a covalent complex, but may refer to a stable non-covalent complex of binding fragments from two different antibodies, humanized binding fragments from two different antibodies, or peptide mimetics derived from binding fragments from two different antibodies. Each binding fragment recognizes a different target or epitope, e.g., a different receptor, e.g., an inhibiting receptor and an activating receptor. Bispecific antibodies normally exhibit specific binding to two different antigens.

“Cutaneous Inflammation” refers to improper regulation of the immune response in the skin or dermis, leading to an infiltrate of inflammatory cells and release of various inflammatory factors, including cytokines. Cutaneous inflammation includes psoriasis, atopic dermatitis, scleroderma, and the like.

As used herein, the term “differentially expressed” refers to the level or activity of a constituent in a first sample (or set of samples) as compared to the level or activity of the constituent in a second sample (or set of samples), where the method used for detecting the constituent provides a different level or activity when applied to the two samples (or sets of samples). Thus, for example, a polypeptide of the invention that is measured at one concentration in a first sample, and at a different concentration in a second sample is differentially expressed in the first sample as compared with the second sample. A marker would be referred to as “increased” in the first sample if the method of detecting the marker indicates that the level or activity of the marker is higher or greater in the first sample than in the second sample (or if the marker is detectable in the first sample but not in the second sample). Conversely, the marker would be referred to as “decreased” in the first sample if the method of detecting the marker indicates that the level or activity of the marker is lower in the first sample than in the second sample (or if the marker is detectable in the second sample but not in the first sample). In particular, a marker is referred to as “increased” or “decreased” in a sample (or set of samples) obtained from a subject (e.g., a psoriasis subject, a subject suspected of having psoriasis, a subject at risk of developing psoriasis) if the level or activity of the marker is higher or lower, respectively, compared to the level of the marker in a sample (or set of samples) obtained from another subject (e.g., a non-psoriasis subject) or subjects or a reference value or range.

Endpoints in activation or inhibition can be monitored as follows. Activation, inhibition, and response to treatment, e.g., of a cell, skin tissue, keratinocyte, physiological fluid, tissue, organ, and animal or human subject, can be monitored by an endpoint. The endpoint may comprise a predetermined quantity or percentage of, e.g., an indicia of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression of gene relating to inflammation, apoptosis, transformation, cell cycle, or metastasis (see, e.g., Knight (2000) Ann. Clin. Lab. Sci. 30:145-158; Hood and Cheresh (2002) Nature Rev. Cancer 2:91-100; Timme, et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126).

To examine the extent of inhibition, for example, samples or assays comprising a given, e.g., protein, gene, cell, or organism, are treated with a potential activator or inhibitor and are compared to control samples without the inhibitor. Control samples, i.e., not treated with antagonist, are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 25%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

“Exogenous” refers to substances that are produced outside an organism, cell, or human body, depending on the context. “Endogenous” refers to substances that are produced within a cell, organism, or human body, depending on the context.

The “fold increase” or “fold decrease” refers to protein expression values that are calculated by the DeCyder v5 (Amersham Biosciences now GE Healthcare) and as described in Alban, et al. (2003) Proteomics 3(1): 36-44.

Typically the calculated average level of modulation of protein expression in psoriatic samples is at least one fold different from normal samples.

A “marker” relates to the phenotype of a cell, tissue, organ, animal, or human subject. Markers are used to detect cells, e.g., during cell purification, quantitation, migration, activation, maturation, or development, and may be used for both in vitro and in vivo studies. An activation marker is a marker that is associated with cell activation.

“Monofunctional reagent” refers, e.g., to an antibody, binding composition derived from the binding site of an antibody, an antibody mimetic, a soluble receptor, engineered, recombinant, or chemically modified derivatives thereof, that specifically binds to a single type of target. For example, a monofunctional reagent may contain one or more functioning binding sites for at least one polypeptide of Table 1. “Monofunctional reagent” also refers to a polypeptide, antibody, or other reagent that contains one or more functioning binding sites for, e.g., for at least one polypeptide of Table 1 and one or more non-functioning binding sites for another type of receptor. For example, a monofunctional reagent may comprise an antibody binding site for at least one polypeptide of Table 1 plus an Fc fragment that has been engineered so that the Fc fragment does not specifically bind to Fc receptor.

“Nucleic acid” refers to deoxyribonucleotides or ribonucleotides and polymers thereof in either single stranded or double-stranded form. The term nucleic acid may be used interchangeably with gene, cDNA, mRNA, oligonucleotide, and polynucleotide. A particular nucleic acid sequence also implicitly encompasses “allelic variants” and “splice variants.”

“Condition” of skin encompasses disorders but also states of skin that are not necessarily classified as disorders, e.g., cosmetic conditions or states of normal physiology. Disorders of a the skin encompass disorders of a cell, where the cell is in the same genetic lineage of the skin, e.g., a precursor cell of dermal keratinocytes where the precursor is committed to becoming a keratinocyte.

As used herein, the terms “psoriatic subject” and “a subject who has psoriasis” refer to a subject who has been diagnosed with psoriasis. The terms “normal subject or tissue” and “a subject who does not have psoriasis” are refer to a subject or tissue from a subject who has not been diagnosed as having psoriasis. Non-psoriasis subjects may be healthy and have no other disease, or they may have a disease other than psoriasis. While human subjects are described herein, it is to be understood that in some embodiments, subject refers to a laboratory animal.

“Sample” refers to a sample from a human, animal, or to a research sample, e.g., a cell, tissue, organ, fluid, gas, aerosol, slurry, colloid, or coagulated material. The “sample” may be tested in vivo, e.g., without removal from the human or animal, or it may be tested in vitro. The sample may be tested after processing, e.g., by histological methods. “Sample” also refers, e.g., to a cell comprising a fluid or tissue sample or a cell separated from a fluid or tissue sample. “Sample” may also refer to a cell, tissue, organ, or fluid that is freshly taken from a human or animal, or to a cell, tissue, organ, or fluid that is processed or stored.

Small molecules are provided for the treatment of physiology and disorders of the skin, e.g., cutaneous inflammation. “Small molecule” is defined as a molecule with a molecular weight that is less than 10 kD, typically less than 2 kD, and preferably less than 1 kD. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules. Small molecule toxins are described (see, e.g., U.S. Pat. No. 6,326,482 issued to Stewart, et al).

“Specifically” or “selectively” binds, when referring to a ligand/receptor, antibody/antigen, or other binding pair, indicates a binding reaction which is determinative of the presence of the protein in a heterogeneous population of proteins and other biologics. us, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity or binding constant that is at least two fold greater, preferably at least ten times greater, more preferably at least 20-times greater, and most preferably at least 100-times greater than the affinity with any other antibody, or binding composition derived thereof. In one embodiment the antibody will have an affinity that is greater than about 10⁹ liters/mol, as determined, e.g., by Scatchard analysis (Munsen, et al. (1980) Analyt. Biochem. 107:220-239).

“Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of a antagonist or agonist of the proteins of Table 1 to a human or animal subject, or to a cell, tissue, physiological compartment, or physiological fluid. “Treatment of a cell, tissue, organ, or subject” encompasses situations where it has not been demonstrated that the antagonist or agonist of the proteins of Table 1 has contacted their respective receptors, or a cell expressing these receptors.

“Therapeutically effective amount” of a therapeutic agent is defined as an amount of each active component of the pharmaceutical formulation that is sufficient to show a meaningful patient benefit, i.e., to cause a decrease in, amelioration of, or prevention of the symptoms of the condition being treated. When the pharmaceutical formulation comprises a diagnostic agent, “a therapeutically effective amount” is defined as an amount that is sufficient to produce a signal, image, or other diagnostic parameter that facilitates diagnosis. Effective amounts of the pharmaceutical formulation will vary according to factors such as the degree of susceptibility of the individual, the age, gender, and weight of the individual, and idiosyncratic responses of the individual (see, e.g., U.S. Pat. No. 5,888,530).

### II. GENERAL

Mammalian skin consists of dermal (inner) and epidermal (outer) layers. The epidermis is made almost entirely of keratinocytes (95%) with other cell types including Langerhans cells and melanocytes. The epidermis is rapidly growing, turning over every seven days in the mouse. In psoriasis, this turnover is shortened to 3-5 days as a result of keratinocyte hyperproliferation.

The present invention is based, in part, upon the proteomic discovery that certain polypeptides were differentially expressed in biological samples from psoriatic patients as compared to biological samples from non-psoriatic patients. Proteomics technologies are particularly adapted for the initial biomarker discovery phase and several proteomics profiling technology platform have emerged for that purpose such as the ProteinChip Biomarker System from Ciphergen or multidimensional LC-MS/MS shotgun approaches.

Plasma from both groups was subjected to liquid chromatograph (LC), 2D-DIGE, and mass spectroscopy (MS). Fluorescence 2-D difference gel electrophoresis (2D-DIGE) is the technology behind the first optimized platform for 2-D difference analysis. 2D-DIGE uses molecular weight- and pI-matched, spectrally resolvable dyes (Cy2, Cy3 and Cy5) to label protein samples prior to 2-D electrophoresis. By using different dyes to separately label proteins isolated from normal and diseased tissues, multiple samples (up to three) can be co-separated and quantitated by three different set of wavelengths. This approach overcomes many of the disadvantages of the traditional 2-D analysis by eliminating the requirement for spot matching.

The ProteinChip Biomarker System uses various chromatographic arrays onto which either intact or pre-fractionated plasma samples are applied and bound proteins are detected by time of flight mass spectrometry. It has been widely used for biomarker discovery (see, e.g., Zhang, Z., R. C. Bast, Jr., et al. (2004). Cancer Research 64(16): 5882-90).

However, identification of the protein of interest is very labor intensive. Multidimensional LC-MS/MS allow identification and quantification of peptides from either intact or pre-fractionated plasma digests. This approach generates large amounts of data and requires vast amount of computational power making it very time consuming and restrictive in the number of samples which can be profiled. On the other hand, two dimensional electrophoresis has been around for over 20 years and has proved invaluable at separating complex mixtures of proteins. Amersham Biosciences has introduced 3 CyDyes (Cy2, Cy3 and Cy5) which are mass and charge matched, therefore allowing up to 3 samples to be co-separated on the same gel eliminating gel to gel variation, the main limitation of 2D gel electrophoresis. In conjunction with a DIGE specific software, DeCyder this technology enables analysis between protein samples. Typically, Cy3 and Cy5 are used to label a protein sample each whereas Cy2 is used to label a pool of all the protein samples to be comparatively analyzed, and serves the role of an internal standard across gels (Alban et al., supra).

Plasma samples from eight psoriatic patients and five non-psoriatic or normal patients was first subjected to depletion of highly abundant proteins, labeling, 2D-DIGE, and MS analysis, as described below. Several proteins were identified as being differentially regulated in psoriatic versus normal plasma, as described in Table 1.

TABLE 1
Differentially expressed proteins

| | | | | Fold change |
|---|---|---|---|---|
| Protein | | | | compared to |
| Identification | Acc # | | Function | normal |
| | | SEQ ID NO | | |
| Apolipoprotein A- | gi\|2914175 | 1 | Apolipoprotein | −1.27, −1.55, −1.24 |
| I Chain A | | | family | (different |
| | | | | isoforms) |
| Apolipoprotein A- | gi\|28762 | 2 | Apolipoprotein | +1.17 |
| IV | | | family | |
| Angiotensinogen | gi\|4557287 | 3 | Body fluid volume | +1.39 |
| | | | and mineral balance - | |
| | | | also protease | |
| | | | inhibitor | |
| Fibrinogen | gi\|2781208 | 4 | Clotting - blood | +1.19, +1.29 |
| Fragment D Chain B | | | coagulation factor | |
| Fibrinogen | gi\|2781209 | 5 | Clotting - blood | +1.15, =1.18 |
| Fragment D Chain C | | | coagulation factor | |
| Fibrinogen, beta | gi\|11761631 | 6 | Clotting - blood | +1.28, +1.23 |
| chain | | | coagulation factor | |
| Fibrinogen, | gi\|4503715 | 7 | Clotting - blood | +1.21, +1.23, |
| gamma chain | | | coagulation factor | +1.25, +1.33 |
| isoform gamma-A | | | | |
| Gamma | gi\|2098509 | 8 | Clotting - blood | +1.5 |
| Fibrinogen 30 Kd | | | coagulation factor | |
| Carboxyl Terminal | | | | |
| Fragment | | | | |
| Clusterin | gi\|32891795 | 9 | Complement | +1.15, +1.18 |
| Complement | gi\|4557385 | 10 | Complement | +1.36 |
| component 3 | | | | |
| Complement | gi\|29565 | 11 | Complement | −1.19, −1.34 |
| component 4 | | | | |
| binding protein, | | | | |
| alpha (C4BPA) | | | | |
| Complement | gi\|14577919 | 12, 13 | Complement | −1.28 |
| component 4A3 | gi\|40737478 | | | |
| Complement | gi\|4502501 | 14 | Complement | +1.28 |
| component 4B | | | | |
| proprotein | | | | |
| | | SEQ ID NO | | |
| | | (polypeptide; | | |
| | | nucleic acid) | | |
| Vitronectin; | gi\|18201911 | 15, 16 | Complement | +1.65, +1.66 |
| serum spreading | gi\|36573 | | | |
| factor; | | | | |
| somatomedin B; | | | | |
| complement S- | | | | |
| protein; epibolin | | | | |
| Hemopexin | gi\|1335098 | 17 | Complement - acute | −1.25, −1.28. −1.57 |
| | | | phase protein | |
| Keratin 1; | gi\|17318569 | 18 | Keratin | −1.25 |
| Keratin-1; | | | | |
| cytokeratin 1 | | | | |
| Keratin 10, type I, | gi\|71528 | 19 | Keratin | −1.22 |
| cytoskeletal | | | | |
| Keratin 10, type I, | gi\|88041 | 20 | Keratin | −1.22 |
| epidermal | | | | |
| Keratin, type II | gi\|1346343 | 21 | Keratin | +1.28 |
| cytoskeletal 1 | | | | |
| (Cytokeratin 1) | | | | |
| (K1) (CK 1) (67 kDa | | | | |
| cytokeratin) | | | | |
| Keratin 2a | gi\|4557703 | 22 | Keratin | +1.28 |
| Alpha-2- | gi\|66932947 | 23 | Protease inhibitor | +1.4 |
| macroglobulin | | | | |
| Retinol binding | gi\|4558179 | 24 | | +1.28 |
| protein 4, plasma | | | | |
| (RBP4) | | | | |

Differential analysis of plasma protein profiles from patients with severe psoriasis (PASI>10) and healthy individuals using a combination of affinity chromatography, 2D-DIGE and LC-MS/MS led to the identification of a list of 21 potential biomarkers for psoriasis (see Table 1). As discussed below, many of these protein changes are in agreement with results from previous studies published in the literature which were carried out in larger cohorts and using various other methods such as immunoassays.

Vitronectin was found in two spots which were 65% and 66% (p-value of 8.9×10⁻⁶ and 9.9×10⁻⁶ respectively) increased in psoriatic plasma compared to normals. In a previous study by Nogita and Kawashima (1992). Arch. Dermatol. Res. 284(5): 315-7, of 45 patients with psoriasis and 14 normal controls, vitronectin was also reported to be significantly higher in plasma from severely diseased patients (PASI>10, n=17, 417.3+/−61.8 ug/ml) than in plasma from controls (290.2+/−43.7 ug/ml) using a sandwich immunoassay. The present results obtained through a totally different blind approach, on a different and smaller population are in agreement with Nogita's study making vitronectin a strong candidate as a biomarker for psoriasis. Nogita also showed that plasma from patients with severe psoriasis also had a higher vitronectin concentration than plasma from patients with a medium form of psoriasis (5<PASI<10, n=11, 323.9+/−71.7 ug/ml) or mild psoriasis (PASI<5, n=17, 280.0+/−54.0 ug/ml), making vitronectin even more attractive as a potential marker of the disease severity.

Similarly, alpha-2 macroglobulin, complement 3 (C3), complement 4B (C4B) and various fibrinogen chains were consistently increased in the plasma of psoriatic patients over the normals. This was also found in a study by Rocha-Pereira, et al. (2004) Br. J. Dermatol. 150(5): 917-28, for potential prognostic markers which included 40 controls and 60 psoriasis patients and showed that these 4 proteins were increased in psoriasis using nephelometry and turbidimetric tests. Moreover, Rocha-Pereira showed that worsening of the disease is associated with elastase/alpha-2 macroglobulin ratios when comparing mild to severe psoriasis. Two additional studies by Orem, et al. (1997). Clin. Chim. Acta 264(1): 49-56, on 39 patients and Vanizor Kural, et al. (2003). Clin. Chim. Acta 328(1-2): 71-82, on 35 patients report an increase in fibrinogen in psoriatic plasma compared to controls. Alpha-2 macroglobulin was also described to be highly increased in a study by Chodorowska, et al. (2004). J. Eur. Acad. Dermatol. Venereol. 18(2): 180-3, in a group of 175 males with medium to severe psoriasis compared to 30 healthy males using an immunoenzymatic method. And the efficacy of various treatments correlated with a considerable decrease of alpha-2 macroglobulin towards the control values. Lastly, C4 levels were significantly higher in patients with psoriasis than in healthy controls in a study by Ozturk, et al. (2001). Immunol. Invest. 30(3): 181-90, using nephelometric detection.

Not only is psoriasis characterized by epidermal hyperproliferation and inflammation, it has also been associated with abnormal plasma lipid metabolism and a high incidence of occlusive cardiovascular events, especially in severe psoriasis. Apolipoproteins play a major role in lipid metabolism and we show an increase in Apo A4 in one spot by 17% (p=0.0023) and a decrease in Apo A1 in 3 spots by 24% (p=0.00068), 27% (p=0.0021) and 55% (p=4.2×10⁻⁵). Determination of plasma lipids and apolipoproteins in psoriatic patients and controls has been examined and reported for many studies and results are controversial. Several studies found no difference in levels of Apo A1 between psoriasis patients and controls. (see, e.g., Uyanik, et al. (2002). Clin. Chem. Lab. Med. 40(1): 65-8; Seckin, et al. (1994). J. Am. Acad. Dermatol. 31(3 Pt 1): 445-9; Imamura, et al. (1990) Nippon Hifuka Gakkai Zasshi Japan. J. Dermatol. 100(10): 1023-8; and Aguilar Martinez, et al. (1989) Dermatologica 179(4): 200-1). On the other hand, Toruniowa, et al. (1990) Przeglad Dermatologiczny 77(2): 96-101 describes higher level of Apo A1 with psoriasis, whereas Seishima, et al. (1994) Br. J. of Dermatol. 130(6): 738-42; Deiana, L. et al. (1992). 68(12): 755-9; and Ferretti, G., R. Alleva, et al. (1994) Acta Dermato Venereologica 74(3): 171-5, show plasma levels of Apo A1 to be significantly lower in the patient group. The present results actually point to 3 spots for three different isoforms of the protein out of 6 in the Apo A1 train of spots to be decreased. The other 3 spots are unchanged between patients and controls. This could account for the discrepancy observed in the literature, demonstrating the power of 2D electrophoresis for distinguishing between different isoforms of a protein.

Psoriasis is also considered to be an angiogenesis related disease, whereby capillary grow in response to growth factors from proliferating cells. As described above, clusterin is increased in two spots by 15% (p=0.0014) and 18% (0.00092) in psoriatic plasma compared to normals. It has also been demonstrated by clusterin antisense oligonucleotides strongly inhibit angiogenesis and induce high level of apoptosis.

Retinol binding protein 4 (RBP4) was found in one spot which increased 28% in psoriatic plasma over the normal samples. RBP4 is the primary plasma transport for vitamin A (retinol), derivatives of which are used to treat severe psoriasis. Acitretin is a systemic retinoid considered one of the treatments of choice for pustular and erythrodermic psoriasis (see, e.g., Lee and Koo (2005) Exp. Opin. Pharmacother. 6(10): 1725-34.) whereas Tazarotene is a topical retinoid indicated for the treatment of plaque psoriasis (Dando and Wellington (2005). American Journal of Clinical Dermatology 6(4): 255-72.). In addition, Rollman and Vahlquist (1985) Arch. Dermatol. Res. 278(1): 17-24, observed an decrease in plasma RBP4 in 28 patients with extensive plaques (>25% of skin surface affected) and pustular or erythrodermic psoriasis over the controls (n=37) but no significant difference between controls and mild to moderate psoriasis (n=79, <25% of skin surface affected).

The above data, reinforces the potential of these proteins as biomarkers for psoriasis. Particularly as a panel, they could constitute a signature for psoriasis, each protein representing an underlying process of the disease such as C3, C4 or fibrinogen for the inflammatory response part, clusterin for the angiogenesis, and Apo A1 for the abnormal lipid metabolism. In addition, vitronectin could indicate worsening of the disease. Furthermore the above 21 proteins can be analyzed in treated patients to check whether they revert to normal with a therapeutic intervention, and therefore could help in monitoring clinical trials.

### III. METHODS OF MEASUREMENT

In the methods of the invention, levels and activity of polypeptides of the invention, polynucleotides of the invention, or cell populations of the invention are measured (or detected) using conventional techniques. The measurement may be quantitative or qualitative. The measurement may be absolute or relative. It should be noted that while one technique may be used to identify the marker, in practice, a different technique may be used to measure the level or activity of the marker. A wide variety of techniques are available, including without limitation mass spectrometry, chromatographic separations, 2-D gel separations, binding assays (e.g., immunoassays), hybridization assays, enzyme assays and competitive inhibition assays, immunofluorescence and cytometry. Any effective method in the art for measuring the level or activity of a polypeptide, polynucleotide or cell population marker of the invention is included in the invention. It is within the ability of one of ordinary skill in the art to determine which method would be most appropriate for measuring a specific marker. Thus, for example, a robust ELISA assay may be best suited for use in a physician's office while a measurement requiring more sophisticated instrumentation may be best suited for use in a clinical laboratory. Regardless of the method selected, it is important that the measurements be reproducible.

Mass spectrometry, which allows direct measurement of analytes with high sensitivity and reproducibility, advantageously can be used to measure polypeptide markers of the invention. A number of mass spectrometric methods are available and could be used to accomplish the measurement. Electrospray ionization (ESI), for example, allows quantification of differences in relative concentration of various species in one sample against another; absolute quantification is possible by normalization techniques (e.g., using an internal standard). Matrix-assisted laser desorption ionization (MALDI) or the related SELDI® technology (Ciphergen, Inc.) also could be used to make a determination of whether a marker was present, and the relative or absolute level of the marker. Moreover, mass spectrometers that allow time-of-flight (TOF) measurements have high accuracy and resolution and are able to measure low abundant species, even in complex matrices like serum or plasma.

For polypeptide markers, quantification can be based on derivatization in combination with isotopic labeling, referred to as isotope coded affinity tags (“ICAT”). In this and other related methods, a specific amino acid in two samples is differentially and isotopically labeled and subsequently separated from peptide background by solid phase capture, wash and release. The intensities of the molecules from the two sources with different isotopic labels can then be accurately quantified with respect to one another.

In addition, one- and two-dimensional gels have been used to separate polypeptides and quantify gel spots by silver staining, fluorescence or radioactive labeling. These differently stained spots have been detected using mass spectrometry, and identified by tandem mass spectrometry techniques.

In certain embodiments, the polypeptide markers are measured using mass spectrometry in connection with a separation technology, such as liquid chromatography-mass spectrometry or gas chromatography-mass spectrometry. It is particularly preferable to couple reverse-phase liquid chromatography to high resolution, high mass accuracy ESI time-of-flight (TOF) mass spectroscopy. This allows spectral intensity measurement of a large number of biomolecules from a relatively small amount of any complex biological material without sacrificing sensitivity or throughput. Analyzing a sample by this method allows the marker (characterized by, for example, the M+H value, or the retention time and mass-to-charge ratio within the given experimental platform) to be determined and quantified.

As will be appreciated by one of skill in the art, many other separation technologies may be used in connection with mass spectrometry. For example, a vast array of separation columns are commercially available. In addition, separations may be performed using custom chromatogaphic surfaces (e.g., a bead on which a marker specific reagent has been immobilized). Molecules retained on the media subsequently may be eluted for analysis by mass spectrometry.

Analysis by liquid chromatography-mass spectrometry produces a mass intensity spectrum, the peaks of which represent various components of the sample, each component having a characteristic mass-to-charge ratio (m/z) and retention time (R.T.) within the given experimental platform. Each polypeptide will have a characteristic M+H value. As one of skill in the art will recognize, there may not be a one- to-one correspondence between components (each with a characteristic m/z and R.T. within the given experimental platform) and the polypeptides having a characteristic M+H value (i.e., the former typically will outnumber the latter). The presence of a peak with the m/z and RT of a marker indicates that the marker is present. The peak representing a marker may be compared to a corresponding peak from another spectrum (e.g., from a control sample) to obtain a relative measurement. Any normalization technique in the art (e.g., an internal standard) may be used when a quantitative measurement is desired. In addition, deconvoluting software is available to separate overlapping peaks. The retention time depends to some degree on the conditions employed in performing the liquid chromatography separation.

The better the mass assignment, the more accurate is the detection and measurement of the marker level in the sample. Thus, the mass spectrometer selected for this purpose preferably provides high mass accuracy and high mass resolution. The mass accuracy of a well-calibrated Micromass TOF instrument, for example, is reported to be approximately 2 mDa, with resolution m/Δm exceeding 5000.

In other embodiments, the level of the polypeptide markers may be determined using a standard immunoassay, such as a sandwich ELISA using matched antibody pairs and chemiluminescent detection. Commercially available or custom monoclonal or polyclonal antibodies are typically used. However, the assay can be adapted for use with other reagents that selectively bind to the marker. Standard protocols and data analysis are used to determine the marker concentrations from the assay data.

A number of the assays discussed above employ an antibody that selectively binds to the marker. An antibody may be identified and produced by any method accepted in the art, as discussed below.

The polypeptide markers of the invention also may be measured using a number of chemical derivatization or reaction techniques known in the art. Reagents for use in such techniques are known in the art, and are commercially available for certain classes of target molecules.

Finally, the chromatographic separation techniques described above also may be coupled to an analytical technique other than mass spectrometry such as fluorescence detection of tagged molecules, NMR, capillary UV, evaporative light scattering or electrochemical detection.

The intracellular levels of polypeptide markers can also be measured. Typical methodologies include protein extraction from a cell or tissue sample, followed by hybridization of a labeled probe (e.g., an antibody) specific for the target protein to the protein sample, and detection of the probe. The label group can be a radioisotope, a fluorescent compound, an enzyme, or an enzyme co-factor. Detection of specific polypeptides may also be assessed by gel electrophoresis or column chromatography, among many other techniques well known to those skilled in the art.

Measurement of the level of a polynucleotide marker may be made by any method known in the art. See, e.g., Sambrook et al., supra; Ausubel et al. (eds.), Current Protocols in Molecular Biology, John Wiley & Sons (1992).

Typical methodologies for RNA detection include RNA extraction from a cell or tissue sample, followed by hybridization of a labeled probe (e.g., a complementary polynucleotide) specific for the target RNA to the extracted RNA, and detection of the probe (e.g., Northern blotting). Detection of specific polynucleotides may also be assessed by gel electrophoresis, column chromatography, direct sequencing, or quantitative PCR, among many other techniques well known to those skilled in the art.

Detection of the presence or number of copies of all or a part of a polypeptide marker gene or polynucleotide of the invention may be performed using any method known in the art. Typically, it is convenient to assess the presence and/or quantity of a DNA or cDNA by Southern analysis, in which total DNA from a cell or tissue sample is extracted, is hybridized with a labeled probe (e.g., a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

Polynucleotide similarity can be evaluated by hybridization between single stranded nucleic acids with complementary or partially complementary sequences. Such experiments are well known in the art.

Cell populations of the invention may be measured and characterized by any method or technique accepted in the art. Flow cytometry, for example, is a widely used means for analyzing the physical and chemical properties of cell populations. Monoclonal antibodies against specific cell-surface or intracellular antigens, conjugated to fluorescent dyes, can be used as probes to detect expression of cellular antigens. After staining a sample with one or more fluorescent probes (either singly or in combination) the cells are conducted by the rapidly flowing stream, one at a time, though a focused laser beam. Information about the cell (e.g., its type, structure, size) can be determined from the fluorescent signal, and the manner in which the cell interacts with and scatters the light from the laser beam. The resulting data is typically compiled in a computer file for subsequent analysis. Flow cytometry also can be used to physically separate cells with particular characteristics (“cell sorting”).

Alternatively, cell populations of the invention may be analyzed using microvolume laser scanning cytometry (MLSC). In MLSC, as with flow cytometry, fluorophore-labeled antibodies specific for cell surface antigens are used to identify, characterize, and enumerate specific leukocyte populations. In one embodiment, the SurroScan® MLSC is used to classify and quantify cell populations. See Dietz et al., U.S. Pat. No. 6,603,537 (issued Aug. 5, 2003); Dietz et al., U.S. Pat. No. 6,687,395 (issued Feb. 3, 2004), Walton et al., supra. The staining reaction can be done with essentially any cell suspension, including whole blood, and assays can be executed in homogeneous mode. Typically, quantitative dilution of the blood-antibody mixture is usually sufficient sample preparation eliminating the need to wash away the reagent, significantly reducing the time needed for sample preparation.

After staining, the cell-antibody mixtures are loaded into optical-quality capillary arrays. The leukocytes of interest distribute throughout the capillary and, in whole blood assays, float to the top of the red cell hematocrit. In order to operate with whole blood, fluorophores that can be excited in the red region (>600 nm) of the spectrum with a HeNe laser, such as Cy5, Cy5.5 and Cy7-APC, are examples. White blood cells isolated following ficoll or erythrocyte-lysis can also be routinely analyzed.

Each capillary in the array is analyzed with the laser-based fluorescence-imaging instrument. In contrast to flow cytometry, the laser scans over stationary cells rather than cells flowing past the laser. A small cylindrical laser spot is scanned across the capillary in one direction while the capillary is translated relative to the optical system in a second direction. Typically three antibody reagents, each with a different fluorescent tag and each detected in a different channel, are used per assay. The capillary is imaged and fluorescent events detected. This is in contrast to flow cytometry where light scatter rather than fluorescence is usually the trigger parameter.

Peaks corresponding to antibody-labeled cells are identified with image processing software that produces a list-mode data file with parameters for every detected cell event. Norton et al., supra. Unlabeled cells i.e., erythrocytes and leukocytes not expressing the target antibodies, are not identified. Intensity data is compensated for spectral overlap, so the resultant values are proportional to the amount of dye-antibody reagent on each cell. The volume of the scan is precisely defined enabling absolute cell counts (cells per μL of blood) to be determined.

Assay panels may be devised to identify and enumerate hundreds of different cell types and cell-associated molecules that are relevant to immune, inflammatory and metabolic processes. In one embodiment, each reagent cocktail typically contains one or two antibodies to the major cell populations—neutrophils, eosinophils, monocytes T-cells, B-cells, NK-cells, and platelets—and one or two antibodies to subsetting antigens which may indicate the functional state, activation state or adhesion characteristics of the population.

### IV. BINDING COMPOSITIONS

Binding compositions provided by the methods of the present invention include may be used to inhibit or induce the expression of the proteins described in Table 1.

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang, et al. (1999) J. Biol. Chem. 274:27371-27378; Baca, et al. (1997) J. Biol. Chem. 272:10678-10684; Chothia, et al. (1989) Nature 342:877-883; Foote and Winter (1992) J. Mol. Biol. 224:487-499; U.S. Pat. No. 6,329,511 issued to Vasquez, et al.).

An alternative to humanization is to use human antibody libraries displayed on phage or human antibody libraries in transgenic mice (Vaughan, et al. (1996) Nature Biotechnol. 14:309-314; Barbas (1995) Nature Medicine 1:837-839; Mendez, et al. (1997) Nature Genetics 15:146-156; Hoogenboom and Chames (2000) Immunol. Today 21:371-377; Barbas, et al. (2001) Phage Display: A Laboratory Manual, Cold Spring Harbor Laboratory Press, Cold Spring Harbor, N.Y.; Kay, et al. (1996) Phage Display of Peptides and Proteins: A Laboratory Manual, Academic Press, San Diego, Calif.; de Bruin, et al. (1999) Nature Biotechnol. 17:397-399).

Single chain antibodies and diabodies are described (see, e.g., Malecki, et al. (2002) Proc. Natl. Acad. Sci. USA 99:213-218; Conrath, et al. (2001) J. Biol. Chem. 276:7346-7350; Desmyter, et al. (2001) J. Biol. Chem. 276:26285-26290; Hudson and Kortt (1999) J. Immunol. Methods 231:177-189; and U.S. Pat. No. 4,946,778). Bifunctional antibodies are provided (see, e.g., Mack, et al. (1995) Proc. Natl. Acad. Sci. USA 92:7021-7025; Carter (2001) J. Immunol. Methods 248:7-15; Volkel, et al. (2001) Protein Engineering 14:815-823; Segal, et al. (2001) J. Immunol. Methods 248:1-6; Brennan, et al (1985) Science 229:81-83; Raso, et al. (1997) J. Biol. Chem. 272:27623; Morrison (1985) Science 229:1202-1207; Traunecker, et al. (1991) EMBO J. 10:3655-3659; and U.S. Pat. Nos. 5,932,448, 5,532,210, and 6,129,914).

The present invention provides a bispecific antibody that can bind specifically to at least two of the molecules described in Table 1, or receptors, thereof (see, e.g., Azzoni, et al. (1998) J. Immunol. 161:3493; Kita, et al. (1999) J. Immunol. 162:6901; Merchant, et al. (2000) J. Biol. Chem. 74:9115; Pandey, et al. (2000) J. Biol. Chem. 275:38633; Zheng, et al. (2001) J. Biol. Chem. 276:12999; Propst, et al. (2000) J. Immunol. 165:2214; Long (1999) Ann. Rev. Immunol. 17:875).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard, et al. (1997) Immunity 7:283-290; Wright, et al. (2000) Immunity 13:233-242; Preston, et al., supra; Kaithamana, et al. (1999) J. Immunol. 163:5157-5164).

Antibodies will usually bind with at least a K_{D} of about 10⁻³ M, more usually at least 10⁻⁶ M, typically at least 10⁻⁷ M, more typically at least 10⁻⁸ M, preferably at least about 10⁻⁹ M, and more preferably at least 10⁻¹⁰M, and most preferably at least 10⁻¹¹ M (see, e.g., Presta, et al. (2001) Thromb. Haemost. 85:379-389; Yang, et al. (2001) Crit. Rev. Oncol. Hematol. 38:17-23; Carnahan, et al. (2003) Clin. Cancer Res. (Suppl.) 9:3982s-3990s).

Polypeptides, antibodies, and nucleic acids, can be conjugated, e.g., to small drug molecules, enzymes, liposomes, polyethylene glycol (PEG), or fusion protein antibodies. Antibodies are useful for diagnostic or kit purposes, and include antibodies coupled, e.g., to dyes, radioisotopes, enzymes, or metals, e.g., colloidal gold (see, e.g., Le Doussal, et al. (1991) J. Immunol. 146:169-175; Gibellini, et al. (1998) J. Immunol. 160:3891-3898; Hsing and Bishop (1999) J. Immunol. 162:2804-2811; Everts, et al. (2002) J. Immunol. 168:883-889).

The invention also provides binding compositions for use as anti-sense nucleic acids or for small interference RNA (siRNA) (see, e.g., Arenz and Schepers (2003) Naturwissenschaften 90:345-359; Sazani and Kole (2003) J. Clin. Invest. 112:481-486; Pirollo, et al. (2003) Pharmacol. Therapeutics 99:55-77; Wang, et al. (2003) Antisense Nucl. Acid Drug Devel. 13:169-189; Cheng, et al. (2003) Mol. Genet. Metab. 80:121-128; Kittler and Buchholz (2003) Semin. Cancer Biol. 13:259-265).

### V. PURIFICATION AND MODIFICATION OF POLYPEPTIDES AND NUCLEIC ACIDS

Polypeptides, e.g., antigens, antibodies, and antibody fragments, and nucleic acids for use in the contemplated method, can be purified by methods that are established in the art. Purification can involve homogenization of cells or tissues, immunoprecipitation, and chromatography. Stability during purification or storage can be enhanced, e.g., by anti-protease agents, anti-oxidants, ionic and non-ionic detergents, and solvents, such as glycerol or dimethylsulfoxide.

Modification of, e.g., peptides, polypeptides, and nucleic acids, includes epitope tags, fluorescent or radioactive groups, monosaccharides or oligosaccharides, sulfate or phosphate groups, C-terminal amides, acetylated and esterified N-groups, acylation, e.g., fatty acid, intrachain cleaved peptide bonds, and deamidation products (see, e.g., Johnson, et al. (1989) J. Biol. Chem. 264:14262-14271; Young, et al. (2001) J. Biol. Chem. 276:37161-37165). Glycosylation depends upon the nature of the recombinant host organism employed or physiological state (see, e.g., Jefferis (2001) BioPharm 14:19-27; Mimura, et al. (2001) J. Biol. Chem. 276:45539-45547; Axford (1999) Biochim. Biophys. Acta 1:219-229; Malhotra, et al. (1995) Nature Medicine 1:237-243).

### VI. THERAPEUTIC COMPOSITIONS AND METHODS

To prepare pharmaceutical or sterile compositions including an antagonist of at least one polypeptide of Table 1, the reagents is mixed with a pharmaceutically acceptable carrier or excipient. Formulations of therapeutic, prophylactic, and diagnostic agents can be prepared by mixing with physiologically acceptable carriers, excipients, or stabilizers in the form of, e.g., lyophilized powders, slurries, aqueous solutions, lotions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

Selecting an administration regimen for a prophylactic or therapeutic depends on several factors, including the serum or tissue turnover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom, et al. (1999) New Engl. J. Med. 341:1966-1973; Slamon, et al. (2001) New Engl. J. Med. 344:783-792; Beniaminovitz, et al. (2000) New Engl. J. Med. 342:613-619; Ghosh, et al. (2003) New Engl. J. Med. 348:24-32; Lipsky, et al. (2000) New Engl. J. Med. 343:1594-1602).

Antibodies, antibody fragments, and cytokines can be provided by continuous infusion, or by doses at intervals of, e.g., one day, one week, or 1-7 times per week. Doses may be provided intravenously, subcutaneously, topically, orally, nasally, rectally, intramuscular, intracerebrally, or by inhalation. A preferred dose protocol is one involving the maximal dose or dose frequency that avoids significant undesirable side effects. A total weekly dose is generally at least 0.05 μg/kg body weight, more generally at least 0.2 μg/kg, most generally at least 0.5 μg/kg, typically at least 1 μg/kg, more typically at least 10 μg/kg, most typically at least 100 μg/kg, preferably at least 0.2 mg/kg, more preferably at least 1.0 mg/kg, most preferably at least 2.0 mg/kg, optimally at least 10 mg/kg, more optimally at least 25 mg/kg, and most optimally at least 50 mg/kg (see, e.g., Yang, et al. (2003) New Engl. J. Med. 349:427-434; Herold, et al. (2002) New Engl. Med. 346:1692-1698; Liu, et al. (1999) J. Neurol. Neurosurg. Psych. 67:451-456; Portielji, et al. (20003) Cancer Immunol. Immunother. 52:133-144). The desired dose of a small molecule therapeutic, e.g., a peptide mimetic, natural product, or organic chemical, is about the same as for an antibody or polypeptide, on a moles/kg body weight basis. The desired plasma concentration of a small molecule therapeutic is about the same as for an antibody, on a moles/kg body weight basis.

An effective amount for a particular patient may vary depending on factors such as the condition being treated, the overall health of the patient, the method route and dose of administration and the severity of side affects, see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK.

Typical veterinary, experimental, or research subjects include monkeys, dogs, cats, rats, mice, rabbits, guinea pigs, horses, and humans.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is derived from the same species as the animal targeted for treatment, thereby minimizing a humoral response to the reagent.

Methods for co-administration or treatment with a second therapeutic agent, e.g., a cytokine, steroid, chemotherapeutic agent, antibiotic, or radiation, are well known in the art (see, e.g., Hardman, et al. (eds.) (2001) Goodman and Gilman's The Pharmacological Basis of Therapeutics, 10^{th} ed., McGraw-Hill, New York, N.Y.; Poole and Peterson (eds.) (2001) Pharmacotherapeutics for Advanced Practice: A Practical Approach, Lippincott, Williams & Wilkins, Phila., PA; Chabner and Longo (eds.) (2001) Cancer Chemotherapy and Biotherapy, Lippincott, Williams & Wilkins, Phila., PA). An effective amount of therapeutic will decrease the symptoms typically by at least 10%; usually by at least 20%; preferably at least about 30%; more preferably at least 40%, and most preferably by at least 50%.

The route of administration is by, e.g., topical or cutaneous application, injection or infusion by intravenous, intraperitoneal, intracerebral, intramuscular, intraocular, intraarterial, intracerebrospinal, intralesional, or pulmonary routes, or by sustained release systems or an implant (see, e.g., Sidman et al. (1983) Biopolymers 22:547-556; Langer, et al. (1981) J. Biomed. Mater. Res. 15:167-277; Langer (1982) Chem. Tech. 12:98-105; Epstein, et al. (1985) Proc. Natl. Acad. Sci. USA 82:3688-3692; Hwang, et al. (1980) Proc. Natl. Acad. Sci. USA 77:4030-4034; U.S. Pat. Nos. 6,350,466 and 6,316,024).

### VII. KITS

The present invention contemplates use of diagnostic kits. Provided are binding compositions, including antibodies or antibody fragments, for the detection the proteins of Table 1, and metabolites and breakdown products thereof, including products resulting from deamidation, limited proteolytic or hydrolytic cleavage, or disulfide bond oxidation or formation. In one embodiment, such binding compositions are bound to a solid support, such as a chip, slide or well, and used as capture reagents. In various embodiments the solid support comprises 1, 2, 3, 4, 5, 6, 7, 8, 9, 10 or more capture reagents, each binding to a different polypeptide of Table 1.

Also provided are proteomic methods of analyzing expression of the proteins of Table 1 to monitor disease progression or success of therapeutic intervention. Typically, the kit will have a compartment containing either at least one polypeptide of Table 1, or an antigenic fragment thereof, a binding composition thereto, or a nucleic acid, e.g., a nucleic acid probe or primer, able to hybridize under stringent conditions to a nucleic acid encoding at least one polypeptide of Table 1.

The kit can comprise, e.g., a reagent and a compartment, a reagent and instructions for use, or a reagent with a compartment and instructions for use. The reagent can comprise full length polypeptide of Table 1, or an antigenic fragment thereof, a binding composition, or a nucleic acid. A kit for determining the binding of a test compound, e.g., acquired from a biological sample or from a chemical library, can comprise a control compound, a labeled compound, and a method of separating free labeled compound from bound labeled compound. The kit may include a solid support comprising one or more capture reagents to detect one or more of the polypeptides of Table 1 and, optionally, instructions for use of the solid support to detect the polypeptides. Capture reagents include, but are not limited to, antibodies or antigen binding fragments thereof.

Conditions enabling stringent hybridization of nucleic acid probes or primers are available (see, e.g., Freeman, et al. (2000) Biotechniques 29:1042-1055; de Silva and Wittwer (2000) J. Chromatogr. B. Biomed. Sci. Appl. 741:3-13; Long (1998) Eur. J. Histochem. 42:101-109; Musiani, et al. (1998) Histol. Histopathol. 13:243-248; Gillespie (1990) Vet. Microbiol. 24:217-233; Giulietti, et al. (2001) Methods 25:386-401; Schweitzer and Kingsmore (2001) Curr. Opin. Biotechnol. 12:21-27; Speel, et al. (1999) J. Histochem. Cytochem. 47:281-288; Tsuruoka and Karube (2003) Comb. Chem. High Throughput Screen. 6:225-234; Rose, et al. (2002) Biotechniques 33:54-56).

Diagnostic assays can be used with biological matrices such as live cells, cell extracts, cell lysates, fixed cells, cell cultures, bodily fluids, including plasma, or forensic samples. Conjugated antibodies useful for diagnostic or kit purposes, include antibodies coupled to dyes, isotopes, enzymes, and metals (see, e.g., Le Doussal, et al. (1991) New Engl. J. Med. 146:169-175; Gibellini, et al. (1998) J. Immunol. 160:3891-3898; Hsing and Bishop (1999) New Engl. J. Med. 162:2804-2811; Everts, et al. (2002) New Engl. J. Med. 168:883-889). Various assay formats exist, such as Real-time PCR, radioimmunoassays (RIA), ELISA, and lab on a chip (U.S. Pat. Nos. 6,176,962 and 6,517,234).

The diagnostic method can comprise contacting a sample from a test subject with a binding composition that specifically binds to at least one polypeptide of Table 1 or nucleic acid encoding at least one polypeptide of Table 1. Moreover, the diagnostic method can further comprise contacting the binding composition to a sample derived from a control subject or control sample, and comparing the binding found with the test subject with the binding found with the control subject or control sample. A “test sample” can be derived from a sample from a subject experiencing psoriasis, both lesional and non-lesional, while a “control sample” can be derived from a sample from a normal (non-psoriatic) subject, or derived from a non-affected skin sample from the subject experiencing cutaneous inflammation. The subject can be, e.g., human, veterinary, experimental, or agricultural. Derived encompasses a biopsy, sample, extract, or a processed, purified, or semi-purified sample or extract.

Alternatively, both test and normal samples, as defined above, can be obtained and subjected to standard mRNA extraction protocols. The mRNA is subsequently reversed transcribed into ssDNA, which is then used for a second DNA strand synthesis. The double strand DNA is then used in real-time PCR, e.g., TaqMan, reactions. As described below, the samples can be analyzed using proteomic analysis methods.

### VIII. METHODS OF SCREENING

In another aspect, the invention provides methods for screening candidate compounds for use as therapeutic agents. In one embodiment, the method comprises screening candidate compounds for those that bind to a polypeptide of the invention, a polynucleotide of the invention, or a cell population of the invention. Candidate compounds that bind to markers can be identified using any suitable method or technique known in the art.

In one embodiment, a candidate compound or a control is contacted with a marker of the invention and the ability of the candidate compound to form stable complexes with the marker is determined (e.g., flow cytometry, immunoprecipitation). The candidate compound, the marker, or an antibody that selectively binds either may be labeled to facilitate detection. The candidate molecule or marker may be immobilized on a solid support (e.g., a bead).

In another embodiment, cells expressing a polypeptide marker are contacted with a candidate compound or a control and the ability of the candidate compound to form stable complexes with the cells is determined. The candidate compound or the marker may be labeled to facilitate detection.

In another embodiment, the method comprises screening candidate compounds for those that have a stimulatory or inhibitory effect on the activity of a marker of the invention comprising comparing the activity of the marker in the presence of the candidate molecule with the activity of the marker in the absence of the candidate molecule (e.g., in the presence of a control).

In another embodiment, the method comprises screening candidate drugs in a clinical trial to determine whether a candidate drug is effective in treating psoriasis. At time t₀, a biological sample is obtained from each subject in population of subjects diagnosed with psoriasis. Next, assays are performed on each subject's sample to measure levels of a marker. In some embodiments, only a single marker is monitored, while in other embodiments, a combination of markers, up to the total number of factors, is monitored. Next, a predetermined dose of a candidate drug is administered to a portion or sub-population of the same subject population. Drug administration can follow any suitable schedule over any time period. In some cases, varying doses are administered to different subjects within the sub-population, or the drug is administered by different routes. At time t₁, after drug administration, a biological sample is acquired from the sub-population and the same assays are performed on the biological samples as were previously performed to obtain measurement values. As before, subsequent sample acquisitions and measurements can be performed as many times as desired over a range of times t₂ to tₙ. In such a study, a different sub-population of the subject population serves as a control group, to which a placebo is administered. The same procedure is then followed for the control group: obtaining the biological sample, processing the sample, and measuring the markers to obtain a measurement chart.

Specific doses and delivery routes can also be examined. The method is performed by administering the candidate drug at specified dose or delivery routes to subjects with psoriasis; obtaining biological samples, such as serum, from the subjects; measuring the level of at least one of the markers in each of the biological samples; and, comparing the measured level for each sample with other samples and/or a standard level or reference level. In one embodiment, the standard level or reference level is obtained by measuring the same marker or markers in the subject before drug administration. Depending upon the difference between the measured and standard levels, the drug can be considered to have an effect on psoriasis. If multiple markers are measured, at least one and up to all of the markers must change, in the expected direction, for the drug to be considered effective. Preferably, multiple markers must change for the drug to be considered effective, and preferably, such change is statistically significant.

As will be apparent to those of ordinary skill in the art, the above description is not limited to a candidate drug, but is applicable to determining whether any therapeutic intervention is effective in treating psoriasis.

In a typical embodiment, a subject population having psoriasis is selected for the study. The population is typically selected using standard protocols for selecting clinical trial subjects. For example, the subjects are generally healthy, are not taking other medication, and are evenly distributed in age and sex. The subject population can also be divided into multiple groups; for example, different sub-populations may be suffering from different types or different degrees of the disorder to which the candidate drug is addressed.

In general, a number of statistical considerations must be made in designing the trial to ensure that statistically significant changes in marker measurements can be detected following drug administration. The amount of change in a marker depends upon a number of factors, including strength of the drug, dose of the drug, and treatment schedule. It will be apparent to one skilled in statistics how to determine appropriate subject population sizes. Preferably, the study is designed to detect relatively small effect sizes.

The subjects optionally may be “washed out” from any previous drug use for a suitable period of time. Washout removes effects of any previous medications so that an accurate baseline measurement can be taken. At time t₀, a biological sample is obtained from each subject in the population. Preferably, the sample is blood, but other biological fluids may be used (e.g., urine). Next, an assay or variety of assays are performed on each subject's sample to measure levels of particular markers of the invention. The assays can use conventional methods and reagents, as described above. If the sample is blood, then the assays typically are performed on either serum or plasma. For other fluids, additional sample preparation steps are included as necessary before the assays are performed. The assays measure values of at least one of the markers of the invention. In some embodiments, only a single marker is monitored, while in other embodiments, a combination of factors, up to the total number of markers, is monitored. The markers may also be monitored in conjunction with other measurements and factors associated with psoriasis (e.g., PASI score). The number of markers whose values are measured depends upon, for example, the availability of assay reagents, biological fluid, and other resources.

Next, a predetermined dose of a candidate drug is administered to a portion or sub-population of the same subject population. Drug administration can follow any suitable schedule over any time period, and the sub-population can include some or all of the subjects in the population. In some cases, varying doses are administered to different subjects within the sub-population, or the drug is administered by different routes. Suitable doses and administration routes depend upon specific characteristics of the drug. At time t₁, after drug administration, another biological sample (the “t₁ sample”) is acquired from the sub-population. Typically, the sample is the same type of sample and processed in the same manner (for example, blood) as the sample acquired from the subject population before drug administration (the “t₀ sample”). The same assays are performed on the t₁ sample as on the to sample t₀ obtain measurement values. Subsequent sample acquisitions and measurements can be performed as many times as desired over a range of times t₂ to tₙ.

Typically, a different sub-population of the subject population is used as a control group, to which a placebo is administered. The same procedure is then followed for the control group: obtaining the biological sample, processing the sample, and measuring the markers to obtain measurement values. Additionally, different drugs can be administered to any number of different sub-populations to compare the effects of the multiple drugs. As will be apparent to those of ordinary skill in the art, the above description is a highly simplified description of a method involving a clinical trial. Clinical trials have many more procedural requirements, and it is to be understood that the method is typically implemented following all such requirements.

Paired measurements of the various markers are thus determined for each subject. The different measurement values are compared and analyzed to determine whether the markers changed in the expected direction for the drug group but not for the placebo group, indicating that the candidate drug is effective in treating psoriasis. The measurement values at time t₁ for the group that received the candidate drug are compared with standard measurement values, preferably the measured values before the drug was given to the group, i.e., at time t₀. Typically, the comparison takes the form of statistical analysis of the measured values of the entire population before and after administration of the drug or placebo. Any conventional statistical method can be used to determine whether the changes in marker values are statistically significant. For example, paired comparisons can be made for each marker using either a parametric paired t-test or a non-parametric sign or sign rank test, depending upon the distribution of the data.

In addition, tests should be performed to ensure that statistically significant changes found in the drug group are not also found in the placebo group. Without such tests, it cannot be determined whether the observed changes occur in all patients and are therefore not a result of candidate drug administration.

As discussed, supra, some of the marker measurement values are higher in samples from psoriasis patients, while others are lower. The nonadjusted p-values shown were obtained by univariate analysis. A significant change in the appropriate direction in the measured value of one or more of the markers indicates that the drug is effective. If only one marker is measured, then that value must increase or decrease to indicate drug efficacy. If more than one marker is measured, then drug efficacy can be indicated by change in only one marker, all markers, or any number in between. In some embodiments, multiple markers are measured, and drug efficacy is indicated by changes in multiple markers. Measurements can be of both markers of the invention and other measurements and factors associated with psoriasis (e.g., measurement of previously known markers reported in the literature). Furthermore, the amount of change in a marker level may be an indication of the relatively efficacy of the drug.

In addition to determining whether a particular drug is effective in treating psoriasis, markers of the invention can also be used to examine dose effects of a candidate drug. There are a number of different ways that varying doses can be examined. For example, different doses of a drug can be administered to different subject populations, and measurements corresponding to each dose analyzed to determine if the differences in the markers before and after drug administration are significant. In this way, a minimal dose required to effect a change can be estimated. In addition, results from different doses can be compared with each other to determine how each marker behaves as a function of dose.

Analogously, administration routes of a particular drug can be examined. The drug can be administered differently to different subject populations, and measurements corresponding to each administration route analyzed to determined if the differences in the markers before and after drug administration are significant. Results from the different routes can also be compared with each other directly.

The broad scope of this invention is best understood with reference to the following examples, which are not intended to limit the inventions to the specific embodiments.

### EXAMPLES

### I. General Methods.

Methods for the diagnosis, prevention, and treatment of inflammatory conditions of the skin in animals and humans are described (see, e.g., Ackerman (1997) Histological Diagnosis of Inflammatory Skin Disease, 2^{nd} ed., Lippincott, Williams, and Wilkins, New York, N.Y.; Gallin, et al. (1999) Inflammation: Basic Principles and Clinical Correlates, 3^{rd} ed., Lippincott, Williams, and Wilkins, New York, N.Y.; Parnham, et al. (1991) Drugs in Inflammation (Agents and Actions Suppl., Vol. 32), Springer Verlag, Inc., New York, N.Y.; Chan (ed.) (2003) Animal Models of Human Inflammatory Skin Diseases, CRC Press, Boca Raton, Fla.; Kownatzki and Norgauer (eds.) (1998) Chemokines and Skin, Birkhauser Verlag, Basel, Switzerland; Kanitakis, et al. (eds.) (1999) Diagnostic Immunohistochemistry of the Skin, Lippincott, Williams, and Wilkins, New York, N.Y.).

Animal models of cutaneous inflammation, and related methods, are available. These methods include use of skin grafts, skin grafts injected with immune cells, subcutaneous injection of immune cells, and use of animals such as various mouse models of psoriasis, in particular xenotransplatation models (see, e.g., Kruger, et al. (1981) J. Clin. Invest., 68:1548-1577; Nickoloff, et al. (1995) Am. J. Pathol. 146:580-588; and Schön (1999) J. Invest. Dermatol. 112:405-410).

Standard methods in molecular biology are described (Maniatis, et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies is described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Standard techniques in cell and tissue culture are described (see, e.g., Freshney (2000) Culture of Animal Cells: A Manual of Basic Technique, 4^{th} ed., Wiley-Liss, Hoboken, N.J.; Masters (ed.) (2000) Animal Cell Culture: A Practical Approach, 3^{rd} ed., Oxford Univ. Press, Oxford, UK; Doyle, et al. (eds.) (1994) Cell and Tissue Culture: Laboratory Procedures, John Wiley and Sons, NY; Melamed, et al. (1990) Flow Cytometry and Sorting Wiley-Liss, Inc., New York, N.Y.; Shapiro (1988) Practical Flow Cytometry Liss, New York, N.Y.; Robinson, et al. (1993) Handbook of Flow Cytometry Methods, Wiley-Liss, New York, N.Y.).

Software packages for determining, e.g., antigenic fragments, signal and leader sequences, protein folding, and functional domains, are available. See, e.g., Vector NTI® Suite (Informax, Inc., Bethesda, Md.); GCG Wisconsin Package (Accelrys, Inc., San Diego, Calif.), and DeCypher® (TimeLogic Corp., Crystal Bay, Nev.); Menne, et al. (2000) Bioinformatics 16:741-742. Public sequence databases were also used, e.g., from GenBank and others.

### II. Plasma Sample Collection

Psoriasis patients (n=45) and normal volunteers (n=30) were consented under a protocol approved by the Stanford Panel on Human Subjects. Psoriasis patients needed to have a Psoriasis Area Severity Index (PASI) of at least 8 to be eligible. They were also required to have a typical lesion at least 1 cm in size suitable for biopsy. The target lesion and the surrounding 5 cm area could not have been treated with any medicated topical formulation for at least 2 weeks prior to obtaining the biopsy. Patients being treated with systemic immunosuppressives including corticosteroids were excluded. Fifty to seventy five ml of peripheral blood was collected in purple topped plastic EDTA-containing tubes and kept at room temperature.

For the present plasma 2D-DIGE profiling study, a subset of patients and normals was selected from the pools described above based on PASI score, age, sex, and ethnicity. Hence the psoriasis group consisted of eight patients (P2, P3, P6, P8, P10, P15, P41 and P42) with a PASI score over 10. The control group consisted of five normal individuals (N2, N4, N11, N16 and N24) matched to the disease group for age, sex (male) and ethnicity (Caucasian).

### III. Depletion of High Abundancy Proteins

Plasma samples were depleted of high abundant proteins using the 4.6×100 mm Multiple Affinity Removal System (Agilent Technologies, Palo Alto, Calif.) following the manufacturer's instructions. This column uses antibodies to remove albumin, IgG, IgA, anti-trypsin, transferrin and haptoglobin from human body fluids. Immunoaffinity chromatography was conducted on a AKTA Explorer (Amersham Biosciences, Piscataway, N.J.). For each sample, both the flowthrough and eluate were collected. Both were acetone precipitated in 4 volumes of cold acetone overnight, resuspended in DIGE buffer (20 mM Tris pH8.8, 7M urea, 2M thiourea, 1% CHAPS, 1% triton X-100, 1% SB 3-10 and 1% ASB 14) and desalted using Protein Desalting Spin Columns (Pierce, Rockford, Ill.) according to the manufacturer's instructions.

Approximately eighty five to ninety percent of the plasma protein content is made up of albumin, IgG, IgA, haptoglobin, transferrin and anti-trypsin which mask the detection of physiologically relevant low abundant proteins. The Multiple Affinity Removal System was used to remove those six high abundant proteins from all plasma samples. FIG. 1 shows a typical SDS-PAGE gel image of proteins in a normal individual plasma before (intact human plasma) and after depletion (flowthrough and eluate). This depletion step greatly enhances the detection of lower abundance proteins.

### IV. Labeling and 2D DIGE of Protein Samples.

Protein concentration was determined using Coomassie® protein assay reagent (Pierce). Typically, 600 pmol of CyDyes were used to label 50 μg of protein sample. The reaction was incubated for 30 minutes on ice in the dark (Unlu et al 1997 Electrophoresis 18:2071-7). After labeling, samples were mixed according to the right experimental design as described in Table 1 and an equal volume of sample buffer (7M urea, 2M thiourea, 1% CHAPS, 1% triton X-100, 1% SB 3-10 and 1% ASB 14, 20 mg/ml DTT and 4% pharmalytes pH3-10 (Amersham Biosciences)) was added. The mixture was completed to 450 μl with rehydration buffer (7M urea, 2M thiourea, 1% CHAPS, 1% triton X-100, 1% SB 3-10 and 1% ASB 14, 2 mg/ml DTT and 1% pharmalytes) before adding to Immobiline DryStrips pH4-7, 24 cm (Amersham Biosciences) for overnight rehydration. Isoelectric Focusing (IEF) was carried out for a total of 40-80 Vhrs (Multiphor, Amersham Biosciences). The IEF strips were equilibrated in buffer containing 5 mg/ml DTT and 45 mg/ml of iodoacetamide and loaded onto 12% Ettan DALT SDS-PAGE gels.

Electrophoresis was carried out for the first half-hour at 25 mA and then at 40 mA until the bromophenol blue was electrophoresed from the gel. Whilst still between the glass plates the SDS-PAGE gels were scanned in the Typhoon 9400. The Cy2, Cy3 and the Cy5 images were collected in a single scan for each gel. Gel image analysis was carried out using the DeCyder software v5 (Amersham Biosciences) and protein spots of interest were robotically picked using the Ettan spot picker (Amersham Biosciences) from a Sypro Ruby stained (Molecular Probes) preparative gel containing 500 μg of sample and robotically digested with trypsin on a Progest (Genomic Solutions).

After depletion, all plasma samples were labeled with Cy2, Cy3 or Cy5 according to the experimental design described in Table 2 and the psoriatic plasma compared to the normals by 2D-DIGE using a pool of all the samples as an internal standard in the Cy2 channel in DeCyder v5. As shown in Table 2, each sample was run individually and all gels were run in triplicate (a, b and c). Analysis of all the gels in DeCyder v5 led to 118 spots being consistently different between the 8 psoriatic plasma samples and the 5 normals using a student's t-test and a cut-off p-value of 0.01. The spread of the differences ranged from −2.15 fold to 2.67 fold and the most significant difference had a t-test p-value of 4.7×10⁻⁹. Spot picking from a preparative gel stained with Sypro Ruby and in-gel digestion followed by LC-MS/MS for those 118 spots of interest resulted in the identification of 21 distinct proteins by two or more peptides. The efficiency of the depletion is reflected in the identities of the proteins found. For example, retinol binding protein 4 (RBP4) was found in one spot which was 28% (+1.28 fold) increased in psoriatic patients over the normals. By opposition, hemopexin was found in three spots which were 25%, 28% and 57% (−1.25, −1.28, and −1.57) decreased in psoriatic compared to normals. All the proteins are summarized in Table 1 based on their functional role. The differentially expressed proteins represent several biological families and functions such as the apolipoprotein family, the complement system, the blood coagulation process, protease inhibitor and acute phase proteins.

TABLE 2
2D-DIGE experimental design used in this study to compare
the plasma of 5 normal patients to 8 psoriatic.

| Total: 24 gels | Cy2 label | Cy3 label | Cy5 label |
|---|---|---|---|
| Gel 1a-1b-1c | Standard | N2 | N4 |
| Gel 2a-2b-2c | Standard | N11 | N16 |
| Gel 3a-3b-3c | Standard | N24 | |
| Gel 4a-4b-4c | Standard | P2 | P3 |
| Gel 5a-5b-5c | Standard | P6 | P8 |
| Gel 6a-6b-6c | Standard | P10 | P15 |
| Gel 7a-7b-7c | Standard | P41 | P42 |

The “standard” sample is made by mixing an equal amount of all the samples, the 5 normals and 8 psoriatic.
The “N Pool” sample is made by mixing an equal amount of the 5 samples from normal individuals.
The “P Pool” sample is made by mixing an equal amount of the 8 samples from psoriatic individuals

### V. Mass Spectrometry of Protein Spots

Mass spectrometry analysis was done on a LCQ Deca Ion Trap (ThermoElectron) with sample introduction with a 48 well Paradigm AS1 autosampler (Michrom Bioresources) and a Paradigm MS4 HPLC system (Michrom Bioresources). The column was self-packed with Vydac C18 resin (5 micron beads, 300 A pores), 10 cm long with a 15 micron tip (New Objectives). The chromatographic separation was done using a linear gradient elution: 8-60% B solvent for 30 minutes (solvent A: 2% acetonitrile, 0.1% formic acid and 0.005% heptafluorobutyric acid, solvent B: 90% acetonitrile, 0.1% formic acid and 0.005% heptafluorobutyric acid).

LC-MS/MS raw files were searched using the Mascot software package against the NCBI non redundant human protein database. Peptide mass tolerance is set at +/−1.5 Daltons and fragment mass tolerance at 0.8 Daltons. Protein identification was based on at least 2 matching peptides.

### VI. Calculation of Data

After 2D electrophoresis, each gel is scanned for Cy2, Cy3 and Cy5. Cy2 was used to label an internal standard, which was made by mixing an equal amount of each sample to be compared in the experiment. The pool standard represented the average of all the samples being analyzed and ensured all the proteins present in the samples were represented. The internal standard was the common element between all the gels. Cy3 and Cy5 were used to label individual samples to be compared.

For each gel, a triplet of images was obtained (one for Cy2, one for Cy3 and one for Cy5). The image triplet was entered into DeCyder. Decyder performed a spot detection and calculated the spots volumes (sum of all the pixels) and the volume ratios (Cy3/Cy2 and Cy5/Cy2).

Then each image triplets was matched to the others using the internal standard in the Cy2 channel. The volume ratios averaged for each group, the normal subjects and the psoriatic subjects.

Many modifications and variations of this invention, as will be apparent to one of ordinary skill in the art, can be made to adapt to a particular situation, material, composition of matter, process, process step or steps, to preserve the objective, spirit, and scope of the invention. All such modifications are intended to be within the scope of the claims appended hereto without departing from the spirit and scope of the invention. The specific embodiments described herein are offered by way of example only, and the invention is to be limited by the terms of the appended claims, along with the full scope of the equivalents to which such claims are entitled; and the invention is not to be limited by the specific embodiments that have been presented herein by way of example.

1-7. (canceled)

8. A method of managing the treatment of psoriasis in a subject comprising:
a) determining first levels of vitronectin and hemopexin in a first plasma sample from the subject;
b) determining second levels of vitronectin and hemopexin in a second plasma sample from the subject,
wherein said first plasma sample is obtained before the second plasma sample; and
c) increasing dosing of a drug to treat the psoriasis if, and only if, an elevated level of vitronectin and a decreased level hemopexin in the second sample compared with the first sample are observed.

9-19. (canceled)

20. The method of claim 8 wherein the levels are determined by 2D DIGE/mass spectrometry analysis or by immunoassay.

21-23. (canceled)


SEQUENCE LISTING (24 sequences)

SEQ ID NO: 1 - protein, 201 aa, Homo sapiens
MLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 2 - protein, 396 aa, Homo sapiens [sequence omitted]

SEQ ID NO: 3 - protein, 485 aa, Homo sapiens
MRKRAPQSEMAPAGVSLRATILCLLAWAGLAAGDRVYIHPFHLVIHNESTCEQLAKANAGKPKDPTFIPAPIQAKTSPVDEKALQDQLVLVAAKLDTEDKLRAAMVGMLANFLGFRIYGMHSELWGVVHGATVLSPTAVFGTLASLYLGALDHTADRLQAILGVPWKDKNCTSRLDAHKVLSALQAVQGLLVAQGRADSQAQLLLSTVVGVFTAPGLHLKQPFVQGLALYTPVVLPRSLDFTELDVAAEKIDRFMQAVTGWKTGCSLMGASVDSTLAFNTYVHFQGKMKGFSLLAEPQEFWVDNSTSVSVPMLSGMGTFQHWSDIQDNFSVTQVPFTESACLLLIQPHYASDLDKVEGLTFQQNSLNWMKKLSPRTIHLTMPQLVLQGSYDLQDLLAQAELPAILHTELNLQKLSNDRIRVGEVLNSIFFELEADEREPTESTQQLNKPEVLEVTLNRPFLFAVYDQSATALHFLGRVANPLSTA

SEQ ID NO: 4 - protein, 328 aa, Homo sapiens
DNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 5 - protein, 319 aa, Homo sapiens
KMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAK

SEQ ID NO: 6 - protein, 491 aa, Homo sapiens
MKRMVSWSFHKLKTMKHLLLLLLCVFLVKSQGVNDNEEGFFSARGHRPLDKKREEAPSLRPAPPPISGGGYRARPAKAAATQKKVERKAPDAGGCLHADPDLGVLCPTGCQLQEALLQQERPIRNSVDELNNNVEAVSQTSSSSFQYMYLLKDLWQKRQKQVKDNENVVNEYSSELEKHQLYIDETVNSNIPTNLRVLRSILENLRSKIQKLESDVSAQMEYCRTPCTVSCNIPVVSGKECEEIIRKGGETSEMYLIQPDSSVKPYRVYCDMNTENGGWTVIQNRQDGSVDFGRKWDPYKQGFGNVATNTDGKNYCGLPGEYWLGNDKISQLTRMGPTELLIEMEDWKGDKVKAHYGGFTVQNEANKYQISVNKYRGTAGNALMDGASQLMGENRTMTIHNGMFFSTYDRDNDGWLTSDPRKQCSKEDGGGWWYNRCHAANPNGRYYWGGQYTWDMAKHGTDDGVVWMNWKGSWYSMRKMSMKIRPFFPQQ

SEQ ID NO: 7 - protein, 437 aa, Homo sapiens
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQAGDV

SEQ ID NO: 8 - protein, 269 aa, Homo sapiens
VQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQAGDV

SEQ ID NO: 9 - protein, 447 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHR

SEQ ID NO: 10 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNLRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 11 - protein, 518 aa, Homo sapiens
TCLPGYVRSHSTQTLTCNSDGEWVYNTFCIYKRCRHPGELRNGQVEIKTDLSFGSQIEFSCSEGFFLIGSTTSRCEVQDRGVGWSHPLPQCEIVKCKPPPDIRNGRHSGEENFYAYGFSVTYSCDPRFSLLGHASISCTVENETIGVWRPSPPTCEKITCRKPDVSHGEMVSGFGPIYNYKDTIVFKCQKGFVLRGSSVIHCDADSKWNPSPPACEPNSCINLPDIPHASWETYPRPTKEDVYVVGTVLRYRCHPGYKPTTDEPTTVICQKNLRWTPYQGCEALCCPEPKLNNGEITQHRKSRPANHCVYFYGDEISFSCHETSRFSAICQGDGTWSPRTPSCGDICNFPPKIAHGHYKQSSSYSFFKEEIIYECDKGYILVGQAKLSCSYSHWSAPAPQCKALCRKPELVNGRLSVDKDQYVEPENVTIQCDSGYGVVGPQSITCSGNRTWYPEVPKCEWETPEGCEQVLTGKRLMQCLPNPEDVKMALEVYKLSLEIEQLELQRDSARQSTLDKEL

SEQ ID NO: 12 - protein, 1744 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAAAAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRDSSTWLTAFVLKVLSLAQEQVGGSPEKLQETSNWLLSQQQADGSFQDPCPVLDRSMQGGLVGNDETVALTAFVTIALHHGLAVFQDEGAEPLKQRVEASISKANSFLGEKASAGLLGAHAAAITAYALTLTKAPVDLLGVAHNNLMAMAQETGDNLYWGSVTGSQSNAVSPTPAPRNPSDPMPQAPALWIETTAYALLHLLLHEGKAEMADQASAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 13 - protein, 534 aa, Homo sapiens
  misc_feature (485)..(485): Xaa can be any naturally occurring amino acid
GQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAAAAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRDSSTWLTAFVLKVLSLAQEQVGGSPEKLQETSNWLLSQQQADGSFQDPCPVLDRSMQGGLVGNDETVALTAFVTIALHHGLAVFQDEGAEPLKQRVEASISKAXSFLGEKASAGLLGAHAAAITAYALTLTKAPVDLLGVAHNNLMAMAQETG

SEQ ID NO: 14 - protein, 1744 aa, Homo sapiens
MRLLWGLIWASSFFTLSLQKPRLLLFSPSVVHLGVPLSVGVQLQDVPRGQVVKGSVFLRNPSRNNVPCSPKVDFTLSSERDFALLSLQVPLKDAKSCGLHQLLRGPEVQLVAHSPWLKDSLSRTTNIQGINLLFSSRRGHLFLQTDQPIYNPGQRVRYRVFALDQKMRPSTDTITVMVENSHGLRVRKKEVYMPSSIFQDDFVIPDISEPGTWKISARFSDGLESNSSTQFEVKKYVLPNFEVKITPGKPYILTVPGHLDEMQLDIQARYIYGKPVQGVAYVRFGLLDEDGKKTFFRGLESQTKLVNGQSHISLSKAEFQDALEKLNMGITDLQGLRLYVAAAIIESPGGEMEEAELTSWYFVSSPFSLDLSKTKRHLVPGAPFLLQALVREMSGSPASGIPVKVSATVSSPGSVPEVQDIQQNTDGSGQVSIPIIIPQTISELQLSVSAGSPHPAIARLTVAAPPSGGPGFLSIERPDSRPPRVGDTLNLNLRAVGSGATFSHYYYMILSRGQIVFMNREPKRTLTSVSVFVDHHLAPSFYFVAFYYHGDHPVANSLRVDVQAGACEGKLELSVDGAKQYRNGESVKLHLETDSLALVALGALDTALYAAGSKSHKPLNMGKVFEAMNSYDLGCGPGGGDSALQVFQAAGLAFSDGDQWTLSRKRLSCPKEKTTRKKRNVNFQKAINEKLGQYASPTAKRCCQDGVTRLPMMRSCEQRAARVQQPDCREPFLSCCQFAESLRKKSRDKGQAGLQRALEILQEEDLIDEDDIPVRSFFPENWLWRVETVDRFQILTLWLPDSLTTWEIHGLSLSKTKGLCVATPVQLRVFREFHLHLRLPMSVRRFEQLELRPVLYNYLDKNLTVSVHVSPVEGLCLAGGGGLAQQVLVPAGSARPVAFSVVPTAATAVSLKVVARGSFEFPVGDAVSKVLQIEKEGAIHREELVYELNPLDHRGRTLEIPGNSDPNMIPDGDFNSYVRVTASDPLDTLGSEGALSPGGVASLLRLPRGCGEQTMIYLAPTLAASRYLDKTEQWSTLPPETKDHAVDLIQKGYMRIQQFRKADGSYAAWLSRGSSTWLTAFVLKVLSLAQEQVGGSPEKLQETSNWLLSQQQADGSFQDLSPVIHRSMQGGLVGNDETVALTAFVTIALHHGLAVFQDEGAEPLKQRVEASISKASSFLGEKASAGLLGAHAAAITAYALTLTKAPADLRGVAHNNLMAMAQETGDNLYWGSVTGSQSNAVSPTPAPRNPSDPMPQAPALWIETTAYALLHLLLHEGKAEMADQAAAWLTRQGSFQGGFRSTQDTVIALDALSAYWIASHTTEERGLNVTLSSTGRNGFKSHALQLNNRQIRGLEEELQFSLGSKINVKVGGNSKGTLKVLRTYNVLDMKNTTCQDLQIEVTVKGHVEYTMEANEDYEDYEYDELPAKDDPDAPLQPVTPLQLFEGRRNRRRREAPKVVEEQESRVHYTVCIWRNGKVGLSGMAIADVTLLSGFHALRADLEKLTSLSDRYVSHFETEGPHVLLYFDSVPTSRECVGFEAVQEVPVGLVQPASATLYDYYNPERRCSVFYGAPSKSRLLATLCSAEVCQCAEGKCPRQRRALERGLQDEDGYRMKFACYYPRVEYGFQVKVLREDSRAAFRLFETKITQVLHFTKDVKAAANQMRNFLVRASCRLRLEPGKEYLIMGLDGATYDLEGHPQYLLDSNSWIEEMPSERLCRSTRQRAACAQLNDFLQEYGTQGCQV

SEQ ID NO: 15 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRAMWLSLFSSEESNLGANNYDDYRMDWLVPATCEPIQSVFFFSGDKYYRVNLRTRRVDTVDPPYPRSIAQYWLGCPAPGHL

SEQ ID NO: 16 - protein, 478 aa, Homo sapiens
  misc_feature (225)..(225): Xaa can be any naturally occurring amino acid
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGXQYWRFEDGVLDPDYPRNISDGFDGIPDNVDAALALPAHSYSGRERVYFFKGKXYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRAMWLSLFSSEESNLGANNYDDYRMDWLVPATCEPIQSVFFFSGDKYYRVNLRTRRVDTVDPPYPRSIAQYWLGCPAPGHL

SEQ ID NO: 17 - protein, 439 aa, Homo sapiens
TPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 18 - protein, 644 aa, Homo sapiens
MSRQFSSRSGYRSGGGFSSGSAGIINYQRRTTSSSTRRSGGGGGRFSSCGGGGGSFGAGGGFGSRSLVNLGGSKSISISVARGGGRGSGFGGGYGGGGFGGGGFGGGGFGGGGIGGGGFGGFGSGGGGFGGGGFGGGGYGGGYGPVCPPGGIQEVTINQSLLQPLNVEIDPEIQKVKSREREQIKSLNNQFASFIDKVRFLEQQNQVLQTKWELLQQVDTSTRTHNLEPYFESFINNLRRRVDQLKSDQSRLDSELKNMQDMVEDYRNKYEDEINKRTNAENEFVTIKKDVDGAYMTKVDLQAKLDNLQQEIDFLTALYQAELSQMQTQISETNVILSMDNNRSLDLDSIIAEVKAQYEDIAQKSKAEAESLYQSKYEELQITAGRHGDSVRNSKIEISELNRVIQRLRSEIDNVKKQISNLQQSISDAEQRGENALKDAKNKLNDLEDALQQAKEDLARLLRDYQELMNTKLALDLEIATYRTLLEGEESRMSGECAPNVSVSVSTSHTTISGGGSRGGGGGGYGSGGSSYGSGGGSYGSGGGGGGGRGSYGSGGSSYGSGGGSYGSGGGGGGHGSYGSGSSSGGYRGGSGGGGGGSSGGRGSGGGSSGGSIGGRGSSSGGVKSSGGSSSVRFVSTTYSGVTR

SEQ ID NO: 19 - protein, 593 aa, Homo sapiens
MSVRYSSSKHYSSSRSGGGGGGGGCGGGGGVSSLRISSSKGSLGGGFSSGGFSGGSFSRGSSGGGCFGGSSGGYGGLGGFGGGSFHGSYGSSSFGGSYGGSFGGGNFGGGSFGGGSFGGGGFGGGGFGGGFGGGFGGDGGLLSGNEKVTMQNLNDRLASYLDKVRALEESNYELEGKIKEWYEKHGNSHQGEPRDYSKYYKTIDDLKNQILNLTTDNANILLQIDNARLAADDFRLKYENEVALRQSVEADINGLRRVLDELTLTKADLEMQIESLTEELAYLKKNHEEEMKDLRNVSTGDVNVEMNAAPGVDLTQLLNNMRSQYEQLAEQNRKDAEAWFNEKSKELTTEIDNNIEQISSYKSEITELRRNVQALEIELQSQLALKQSLEASLAETEGRYCVQLSQIHAQISALEEQLQQIRAETECQNTEYQQLLDIKIRLENEIQTYRSLLEGEGSSGGGGRGGGSFGGGYGGGSSGGGSSGGGYGGGHGGSSGGGYGGGSSGGGSSGGGYGGGSSSGGHGGGSSSGGHGGSSSGGYGGGSSGGGGGGYGGGSSGGGSSSGGGYGGGSSSGGHKSSSSGSVGESSSKGPRY

SEQ ID NO: 20 - protein, 561 aa, Homo sapiens
MSVRYSSSSKQFSSSRSGGGGGGGSSLRISSSKGSLGGGFSSGGFSGGSFSRGSSGGGCFGGSSGGYGGLGGFGGGSFRGSYGSSSFGGSYGGSFGGGSFGGGSFGGGSFGGGGFGGGGFGGGFGGGFGGDGGLLSGNEKVTMQNLNDRLASYLDKVRALEESNYELEGKIKERYDQHGNSRQGEPRDYSKYYKTIDDLKNQILNLTTDNANILLQIDNARLAADDFRLKYENEVALRQSVEADINGLRRVLDELTLTQADLEMQIESLTEELAYLKKNREEEMKHLRNVSTGDVNVEMNAAPGVDLTQLLNNMRNQYEQLAEQNRKDAEAWFNEKSKELTTEIDNNIEQISSYKSEITELRRNVQALEIELQSQLALKQSLEASLAETEGRYCVQLSQIQAQISALEEQLQEIRAETECQNTEYQQLTDIKIRLENEIQTYRSLLEGEGSSGGGGRGGGSFGGGYGGGSSGGGSSGGGYGGGSSSGGHGGSSSGGYGGGSFGGGGGGYGGGSSGGGSSSGGGYGGGSSSGGGYGGGSSSGGHKSSSSGSVGESSSKGPRY

SEQ ID NO: 21 - protein, 644 aa, Homo sapiens
MSRQFSSRSGYRSGGGFSSGSAGIINYQRRTTSSSTRRSGGGGGRFSSCGGGGGSFGAGGGFGSRSLVNLGGSKSISISVARGGGRGSGFGGGYGGGGFGGGGFGGGGFGGGGIGGGGFGGFGSGGGGFGGGGFGGGGYGGGYGPVCPPGGIQEVTINQSLLQPLNVEIDPEIQKVKSREREQIKSLNNQFASFIDKVRFLEQQNQVLQTKWELLQQVDTSTRTHNLEPYFESFINNLRRRVDQLKSDQSRLDSELKNMQDMVEDYRNKYEDEINKRTNAENEFVTIKKDVDGAYMTKVDLQAKLDNLQQEIDFLTALYQAELSQMQTQISETNVILSMDNNRSLDLDSIIAEVKAQNEDIAQKSKAEAESLYQSKYEELQITAGRHGDSVRNSKIEISELNRVIQRLRSEIDNVKKQISNLQQSISDAEQRGENALKDAKNKLNDLEDALQQAKEDLARLLRDYQELMNTKLALDLEIATYRTLLEGEESRMSGECAPNVSVSVSTSHTTISGGGSRGGGGGGYGSGGSSYGSGGGSYGSGGGGGGGRGSYGSGGSSYGSGGGSYGSGGGGGGHGSYGSGSSSGGYRGGSGGGGGGSSGGRGSGGGSSGGSIGGRGSSSGGVKSSGGSSSVRFVSTTYSGVTR

SEQ ID NO: 22 - protein, 639 aa, Homo sapiens
MSCQISCKSRGRGGGGGGFRGFSSGSAVVSGGSRRSTSSFSCLSRHGGGGGGFGGGGFGSRSLVGLGGTKSISISVAGGGGGFGAAGGFGGRGGGFGGGSSFGGGSGFSGGGFGGGGFGGGRFGGFGGPGGVGGLGGPGGFGPGGYPGGIHEVSVNQSLLQPLNVKVDPEIQNVKAQEREQIKTLNNKFASFIDKVRFLEQQNQVLQTKWELLQQMNVGTRPINLEPIFQGYIDSLKRYLDGLTAERTSQNSELNNMQDLVEDYKKKYEDEINKRTAAENDFVTLKKDVDNAYMIKVELQSKVDLLNQEIEFLKVLYDAEISQIHQSVTDTNVILSMDNSRNLDLDSIIAEVKAQYEEIAQRSKEEAEALYHSKYEELQVTVGRHGDSLKEIKIEISELNRVIQRLQGEIAHVKKQCKNVQDAIADAEQRGEHALKDARNKLNDLEEALQQAKEDLARLLRDYQELMNVKLALDVEIATYRKLLEGEECRMSGDLSSNVTVSVTSSTISSNVASKAAFGGSGGRGSSSGGGYSSGSSSYGSGGRQSGSRGGSGGGGSISGGGYGSGGGSGGRYGSGGGSKGGSISGGGYGSGGGKHSSGGGSRGGSSSGGGYGSGGGGSSSVKGSSGEAFGSSVTFSFR

SEQ ID NO: 23 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDDEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 24 - protein, 180 aa, Homo sapiens
CAVSSFRVKENFDKARFSGTWYAMAKKDPEGLFLQDNIVAEFSVDETGQMSATAKGRVRLLNNWDVCADMVGTFTDTEDPAKFKMKYWGVASFLQKGNDDHWIVDTDYDTYAVQYSCRLLNLDGTCADSYSFVFSRDPNGLPPEAQKIVAQRQEELCLAAQYRLIVHNGYCDGRSERNLL